(19) United States
(12) Patent Application Publication
(10) Pub. No.: US 2016/0213866 A1
(43) Pub. Date: Jul. 28, 2016

(54) Ultrasonic vaporizing element

(71) Applicant: William Tan, San Gabriel, CA (US)

(72) Inventor: William Tan, San Gabriel, CA (US)

(21) Appl. No.: 15/004,920

(22) Filed: Jan. 23, 2016

Related U.S. Application Data
(60) Provisional application No. 62/106,852, filed on Jan. 23, 2015.
(60) Provisional application No. 62/142,464, filed on Apr. 2, 2015.

(51) Int. Cl. A61M 15/06; A61M 15/00; A24F 47/00; A61M 11/00

(52) U.S. Cl. CPC A61M 15/06; A61M 11/005; A61M 15/0021; A24F 47/002; A61M 2205/8206

(57) ABSTRACT

An electronic vaporization device, used for the purpose of inhalation of vapor created by using an ultrasonic signal generator to vaporize a solid or liquid; the apparatus also employs a Langevin transducer, a horn, and a probe, which is attached to the horn and is bent to optimize vibrational amplitude/displacement at the tip.

This application claims the benefit of United States Provisional Patent Appl. No. 62/106,852, filed on Jan. 23, 2015, and U.S. Provisional Patent Appl. No. 62/142,464, filed on Apr. 2, 2015, which are incorporated by reference in entirety.

### BACKGROUND OF THE INVENTION

1. Field of the Invention

An improvement for an ultrasonic vaporizing element for an electronic cigarette.

2. Description of Related Art

In recent years, portable electronic vaporizers, or commonly known as electronic cigarettes or “e-cigs” have gained popularity among users who vaporize herbal extracts to inhale the vapors emitted when the extracts are heated. This action is often referred to as “vaping.” An electronic vaporizer uses one or more batteries to power a heating element, which then heats up a small amount of the vaping liquid at an appropriate temperature to convert the liquid to vapor, which the user then inhales. Vaping liquids are typically solutions of propylene glycol, glycerol, or both, plus nicotine and flavorant chemicals. Medical marijuana users also add cannabis extracts to the mixture. Some E-cigarettes are also designed to allow for vaporization of solid herbal extracts. Various types of liquid and solid herbal extracts are available and are commonly referred to as “e-juice,” “essential oil,” “butter,” “concentrate” or “wax.”

A common heating element is Nichrome resistance wire, which is a non-magnetic alloy of nickel, chromium, and often iron. A typical device will contain a rechargeable battery (usually lithium-ion), which is connected to a circuit board that has an On/Off switch. When the switch is turned on, the current from the battery will flow to the heating element to generate heat and causes the herbal extract that is placed on or close to the heating element to vaporize. The user will then inhale the vapor via a mouthpiece. A drawback of using Nichrome as a heat source is that nickel alloys and compounds are classified as carcinogenic to humans: www.nipera.org/WorkplaceGuide/WorkplaceSurveillance/CarcinogenicClassifications.aspx.

Another disadvantage of the heating system of such vaporizers is that after several uses, the heating element will be covered with residue from the extract. Over time, the residue will build up and causes a drop in the heater's efficiency. At such point, the device will be rendered inoperable, and the user will need to replace or repair the heating element.

Health issues associated with “vaping” have also been reported by The New England Journal of Medicine (www.nejm.org/doi/full/10.1056/NEJMc1413069). At heated temperatures, formaldehyde, a known carcinogen, is released.

Ultrasonic atomization: a more effective method for vaporizing herbal extracts is thus explored via ultrasonic atomization. Ultrasonic atomization or nebulization has been studied for decades and much has been written about these subjects and shall not be elaborated in detail here. Briefly, atomization occurs through the rapid mechanical upward and downward motion of an ultrasonic tip, which causes a film of liquid to form into standing capillary waves.

When the amplitude of the capillary wave peaks above what is required for stability of the system, the liquid at the peak crests breaks away in the form of droplets. The phenomenon known as cavitation occurs at higher energy levels. Microscopic gas bubbles in a liquid will be forced to oscillate due to the applied sound wave. At such high intensity, bubbles will grow in size and rapidly collapse or implode. This disintegration of the liquid also results in droplet formation. Ultrasonic atomization is often used in automotive spray painting, in humidifiers, and coatings for fuel cells, wafers and solar panels.

Ultrasonic sound waves are generally created by piezoelectric or magnetostrictive transducers. Piezoelectric transducers utilize the piezoelectric property of a material to convert electrical energy directly into mechanical energy. Magnetostrictive transducers utilize the magnetostrictive property of a material to convert the energy in a magnetic field into mechanical energy.

Investigation into the suitable ultrasonic frequency and amplitude is made using an Ultrasonic Processor, such as the Hielscher UP100H model; this is lab equipment used for sonochemistry, which is the study of emulsifying, dispersing, dissolving and cell disruption of liquids. A frequency range of 20-40 kHz and a displacement amplitude of at least 10 micrometer at the vibrating tip is observed to sufficiently atomize a thin layer of vaping liquid with comparable vapor volume as that generated by traditional electronic cigarettes.

However, using a lab ultrasonic processor will be impractical as a portable vaporizer due to the size, weight and high power requirement. Another important consideration is the method of containing and delivering the vaping liquid to the vibrating ultrasonic tip (sonotrode or horn). Using a separate beaker like that used in conjunction with a lab ultrasonic processor will not be leak proof or practical.

Nebulizers: other types of vaporizers in the medical industry exist whereby liquids are atomized using ultrasonic sound waves. These types of vaporizers are also called nebulizers, which is a drug delivery device used to administer medication in the form of a mist inhaled into the lungs to treat cystic fibrosis, asthma, COPD and other respiratory diseases. Different types of ultrasonic nebulizers are described below:

Ultrasonic wave nebulizers—these have an electronic oscillator generate a high frequency ultrasonic wave, which causes the mechanical vibration of a Mist Transducer. This transducer is comprised of a ring-shaped piezoelectric element attached to a metal plate, which amplifies the vibration. The metal plate is in contact with a liquid reservoir, and its high frequency vibration is sufficient to produce a vapor mist. An example of such a type of Mist Transducer is the SMIST15F28RR111 model produced by Steiner and Martins, Inc. of Florida, USA. Examples of such nebulizers are: Omron NE-U17 and Beurer Nebulizer IH30. These nebulizers are often larger table top devices requiring plug-in power.

Vibrating mesh technology (VMT): a metallic mesh/membrane with 1000-7000 laser drilled holes vibrates at the top of the liquid reservoir, and thereby pressures out a mist of very fine droplets through the holes. This technology is more efficient than having a vibrating piezoelectric element at the bottom of the liquid reservoir, and thereby allows for smaller and more portable designs. The mesh can be vibrated with the same ring-shaped piezoelectric element attached to the mesh, or it can be vibrated by a Langevin type transducer placed against the mesh; some examples are Pari eFlow, Respironics i-Neb, Beurer Nebulizer IHSO, Aerogen Aeroneb and Omron MicroAir products.

Sandwich type ultrasonic transducers, also called bolt-clamped or Langevin transducers, are well known and established for the production of high intensity ultrasonic motion. In United Kingdom Patent No. 145,691, issued in 1921, P. Langevin inventor, a sandwich of piezoelectric material positioned between metal plates is described to generate high intensity ultrasound. Sandwich transducers utilizing a bolted stack transducer tuned to a resonant frequency and designed to a half wavelength of the resonant frequency are described in United Kingdom Patent No. 868,784.

Regardless of the types of nebulizer technology, these devices are inappropriate for vaping liquids or solids used in electronic cigarettes. The drugs used for nebulizing are often water-like in viscosity (water=1 cSt), whereas vaping liquids are often more than 50 cSt, depending on the types and concentration of glycol, glycerol and nicotine in the mixture. Bubbling is observed when vaping liquids are used in medical nebulizers, but the intensity of the vibration is insufficient to cause atomization.

An obvious solution to the aforementioned problem is to use a higher-powered transducer. However, this is unsuitable for the following reasons:

1. Size—The ring-shaped transducers used in ultrasonic wave or VMT nebulizers are often 1.5 cm to 4 cm in diameter. For such transducers to be powerful enough to atomize viscous vaping liquids, the transducer has to be significantly bigger for higher amplification. This is apparently unsuitable to be incorporated in a pocket-sized vaporizer.

2. Power—A higher power input is required for powering a larger transducer, which will be unsuitable for a portable device running on battery.

3. Noise—A VMT nebulizer utilizing a Langevin transducer will generate a very noticeable high-pitch noise if the transducer sonotrode or tip vibrates at the desired amplitude of more than 10 micrometer against metal or any hard objects. This rules out any transducer-to-metal mesh vibration mechanism.

Another consideration is the cleanability of the device. Vaping liquids containing cannabis extracts, or solid cannabis extracts are thick, sticky and do not dissolve in water or detergent. At room temperature, these liquids and are difficult to wash off without acidic solvents. Thus, any liquid tank in the portable device should preferably be replaceable and disposable.

However, existing nebulizers using VMT often incorporate the mesh and transducer directly onto the removable liquid tanks and are not intended to be discarded after short uses. The preferred configuration should be to separate the tank from the transducer or vibrating source so that the tank itself can be cheaply replaced. This presents a challenge with the appropriate methodology of delivering the vaping liquid from a removable tank to the transducer that is permanently attached to the device, and yet is leak proof when transported.

From the preceding descriptions, it is apparent that the devices currently being used have significant disadvantages. Thus, important aspects of the technology used in the field of invention remain amenable to useful refinement.

### SUMMARY OF THE INVENTION

An apparatus for ultrasonic atomizing of a liquid in a container, comprising: a housing with a power source; the housing has a mouthpiece and the container with the liquid for atomizing; the container has a wick for drawing the liquid to a probe on an ultrasonic transducer, which is located in the housing; the probe engages the wick and allows for a displacement of greater than 10 micrometer; the ultrasonic transducer is connected to the power source and a signal generator; whereby when activated, the ultrasonic transducer vibrates the probe, such that the liquid from the wick is atomized, and vapor of the atomized liquid exits the mouthpiece.

An apparatus for ultrasonic atomizing of a substance in a container with a soft bottom membrane and at least one rigid side wall, comprising: a housing with a power source; the housing has a mouthpiece and the container with the substance for atomizing; the housing has an ultrasonic transducer, which contacts the soft bottom membrane of the container; the ultrasonic transducer is connected to the power source and a signal generator; whereby when activated, the ultrasonic transducer vibrates the soft bottom membrane of the container such that the substance is atomized, and vapor of the atomized substance exits the mouthpiece.

An apparatus for ultrasonic atomizing of a substance in a container, comprising: a housing with a power source; the housing has a mouthpiece and the container with the substance for atomizing; the housing has an ultrasonic transducer, which is integrated with the container; the ultrasonic transducer is connected to the power source and a signal generator; whereby when activated, the ultrasonic transducer vibrates the container such that the substance is atomized, and vapor of the atomized substance exits the mouthpiece.

The power source can be an internal battery or an external power source; the ultrasonic transducer can be activated by an activation switch or button; the container can be removable from the housing; the ultrasonic transducer can have multiple piezoelectric elements, a transduction portion, an anvil bearing member and a sonotrode amplification member; the ultrasonic transducer can be acoustically coupled; the housing can have acoustic isolators to dampen vibrations emitted from the ultrasonic transducer to the housing.

The invention presents an apparatus and method to nebulize vaping liquids or solids using an ultrasonic transducer to produce vibrations along a longitudinal axis at a predetermined frequency, such that the vibratory energy at the forward end of the sonotrode is effectively transferred to the vaping medium to cause nebulization.

The present invention includes a replaceable vaping liquid tank, containing a porous absorbent material interfacing the forward end of the sonotrode and vaping liquid so that the liquid is delivered to the sonotrode for nebulizing in a controlled manner. In a second embodiment, a removable container with a soft membrane bottom interfaces the sonotrode so that vaping solids placed in the container can be nebulized by the transfer of vibratory energy from the forward end of the sonotrode, through the soft membrane, to the vaping solids. In a third embodiment, the sonotrode is configured to comprise a removable metal container at its forward end. The metal container is adapted to the sonotrode to receive the ultrasonic transmission waveguide, such that vaping solids placed in the container can be nebulized when exposed to vibratory energy. The present invention introduces such refinements. In its preferred embodiments, the present invention has several aspects or facets that can be used independently, although they are preferably employed together to optimize their benefits. All of the foregoing operational principles and advantages of the present invention will be more fully appreciated upon consideration of the following detailed description, with reference to the drawings.

### BRIEF DESCRIPTION OF THE DRAWINGS

FIG. 1 is a longitudinal cross-sectional view of a first embodiment of the nebulizer with a removable liquid tank according to the invention.

FIG. 2 is a longitudinal cross-sectional view of a second embodiment of the nebulizer with a removable soft membrane container according to the invention.

FIG. 3 is a longitudinal cross-sectional view of a third embodiment of the nebulizer with a removable solid tank container incorporated into the sonotrode according to the invention.

### PARTS LISTING

10 nebulizer
12 body
14 mouthpiece
20 ultrasonic transducer assembly or transducer assembly
22 piezoelectric elements
24 transduction portion
26 “anvil” or bearing member
28 amplification member or “sonotrode”
30 velocity transformer
32 bolt
38, 40 acoustic isolators
42, 44 electric current supply wires
50 high frequency signal generator
52 battery or power source
56 activation actuator or on/off switch
58 input power jack
60 circuit board
70 tank assembly
72 tank
74 vaping liquid or substance
76 soft absorbent material or “wick”
78 stopper
80 removable container
82 container side wall
84 soft bottom membrane
90 container
92 side wall of container
94 container bottom DESCRIPTION OF THE PREFERRED EMBODIMENTS

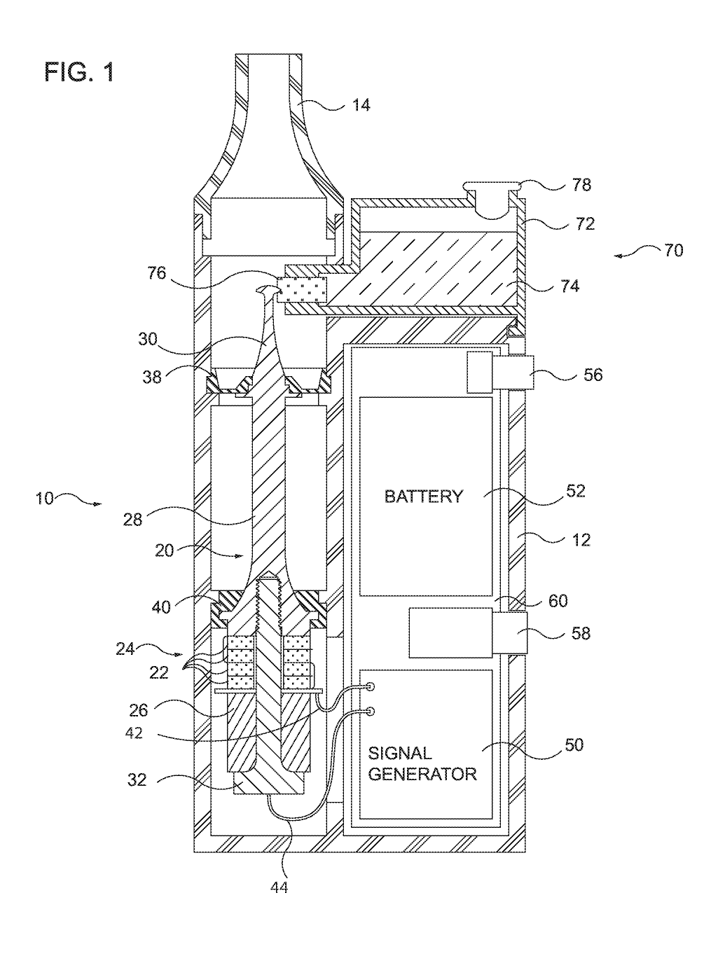

Referring to FIGS. 1-3, there is illustrated several preferred embodiments for the vaporizing element for a certain type of e-cigarette or “e-cig,”—each said embodiment employs an ultrasonic signal generator to vaporize the e-juice (or material to be vaporized, including without limitation “wax,” gel or solid vaporizable substances) from the e-juice container or tank.

This invention uses and employs ultrasonic signals and vibrations to cause atomization of e-juice, such as a nebulizer. There are existing nebulizers, such as: (http://www.omron-healthcare.com/eu/en/our-products/respiratory-therapy/microair-u22), but these nebulizers work with thin liquids that are water-like (viscosity=1 Cst), and are not powerful enough for viscous liquids >10 Cst.

The inventor has discovered such a suitable frequency is about 30 kHz, and the amplitude of the vibrating probe in contact with liquid at >10 micrometer. A higher displacement will create more vapor output. Lower frequencies can also atomize, but the vapor particles will be too large. It is presumed that no prior art vaporizers or nebulizers exist that work in the desired frequency/amplitude range. Ultrasonic lab equipment for sonochemistry exists for cavitation of liquids and is powerful enough for atomizing e-juice.

However, these existing lab equipment devices are high-powered and often have very long ultrasonic “horns” or probes that amplify the signal. This setup makes it ineffective for a portable device.

The discovery of shaping a transducer horn so that amplitude can be increased has been important; it is L-shaped (like dental scaling tips). This takes up less space with similar vibrational displacement achieved at the end of the probe. Due to the vibration energy created, especially at the end of the probe, heat is generated. This can reach >70° C., which means that the device can also be used for solids or waxes such as THC. THC melts at 66° C. and turns into a liquid, which can then be ultrasonically atomized.

A preferred embodiment of an ultrasonic atomizing/nebulizing device, which generally comprises: an ultrasonic generator (typical, existing), powered by a DC power supply (can be battery or DC from an AC/DC converter); an ultrasonic generator drives a Langevin transducer; a horn, a part of the transducer, is a typical component of a Langevin transducer to amplify the ultrasonic signal, but not sufficient to atomize viscous liquid; a metal probe is attached to horn; a probe is bent to allow for greater movement of the probe tip so as to create a displacement of >10 micrometer; a tip touches a cotton wick, which draws fluid from a removable tank; and vapor of the atomized substance or liquid exits the mouthpiece.

In FIGS. 1 to 3, the nebulizer 10 according to the invention comprises: a body 12, comprising a top portion mouthpiece 14, and encloses an ultrasonic transducer assembly 20 constituted by a stack of piezoelectric elements 22 which are supplied with electric current by supply wires 42 and 44 connected to a high frequency signal generator 50, which is an integral component on circuit board 60 which also includes primarily an on/off switch 56, a battery power source 52, and an input power jack 58. The transducer assembly 20 is held within the nebulizer housing 12 with acoustic isolators 40 and 38, so as to dampen the vibrations emitted from the transducer assembly 20 to the housing 12. Acoustic isolator 38 also acts as a seal to prevent nebulized droplets from straying into other components of the nebulizer 10.

The transducer assembly 20, which is known as a “Langevin stack,” generally includes a transduction portion 24, a bearing member or “anvil” 26, and an amplification member or “sonotrode” 28. The bearing member or “anvil” 26 is connected to the proximal end of transduction section 24, and the sonotrode 28 is connected to the distal end of transduction portion 24. The anvil 26, sonotrode 28 are preferably fabricated from titanium, aluminum, stainless steel, or any other suitable material. Sonotrode 28 and anvil 26 have a length determined by a number of variables, including the thickness of the transduction section 24, the density and modulus of elasticity of material used in sonotrode 28, anvil 26 and the resonant frequency of the transducer assembly 20. The sonotrode 28 may be tapered inwardly from its proximal end to its distal end to amplify the ultrasonic vibration amplitude as velocity transformer 30, or alternately may have no amplification.

The piezoelectric elements 22 may be fabricated from any suitable material, such as, for example, lead zirconate-titanate, lead meta-niobate, lead titanate, or other piezoelectric crystal material. The piezoelectric elements 22 have a bore extending through the center and are electrically coupled to wires 42 and 44, and electrically connected to the signal generator 50 on circuit board 60. The circuit board 60 also comprises a power source in the form of a rechargeable battery 52, an on/off switch 56 and a power jack 58 for accepting an external power source for charging the rechargeable battery 52 or to power the circuit board 50 in the absence of battery 52. Alternatively, the power jack 58 can be omitted if the battery 52 is removable from the nebulizer 10 for charging externally, or the nebulizer 10 is powered only by plug-in power through the power jack 58 without a battery power source.

The piezoelectric elements 22 are conventionally held in compression between anvil 26 and sonotrode 28 by a bolt 32. The bolt 32 preferably has a head, a shank, and a threaded distal end. The bolt 32 is inserted from the proximal end of anvil 26 through the bores of anvil 26 and piezoelectric elements 22. The threaded distal end of the bolt 32 is screwed into a threaded bore in the proximal end of sonotrode 28. A removable mouthpiece 14 is attached to the nebulizer housing 12 to direct the vapor generated for inhalation by the user.

In order for the transducer assembly 20 to deliver energy all components of transducer assembly 20 must be acoustically coupled. The components of the transducer assembly 20 are preferably acoustically tuned such that the length of any assembly is an integral number of one-half wavelengths (nλ/2), where the wavelength λ is the wavelength of a pre-selected or operating longitudinal vibration drive frequency f_{d} of the acoustic assembly 20, and where n is any positive integer. It is also contemplated that the acoustic assembly 20 may incorporate any suitable arrangement of acoustic elements.

Referring to FIGS. 1 to 3, wires 42 and 44 transmit the electrical signal from the signal generator 50 to the piezoelectric elements 22 of the transducer assembly 20. The signal generator 50 is in turn electrically powered by a battery 52, and drives the circuit board 60 that primarily includes the signal generator 50, on/off switch 56 and power input jack 58. The piezoelectric elements 22 are energized by an electrical signal supplied from the generator 50 in response to the on/off switch 56 to produce an acoustic standing wave in the transducer 20. The electrical signal causes disturbances in the piezoelectric elements 22 in the form of repeated small displacements resulting in large compression forces within the material. The repeated small displacements cause the piezoelectric elements 22 to expand and contract in a continuous manner along the axis of the voltage gradient, producing longitudinal waves of ultrasonic energy. The ultrasonic energy is transmitted through the acoustic assembly 20 to the velocity transformer 30. Vaping medium or substance in contact with the distal end of the velocity transformer 30 is energized or agitated to the point of atomization or cavitation, resulting in nebulization of the vaping medium.

In FIG. 1 of the first embodiment, a removable tank assembly 70 is attached to the housing 12 to store an amount of vaping liquid 74 within the tank 72. On one opening end of the tank 72, a soft absorbent material or “wick” 76 is adapted to draw a small amount of vaping liquid 74 to the distal end of the velocity transformer 30. The wick 76 is made from materials, including without limitation: cotton, fiberglass, ceramic fiber or any material that is absorbent and acoustically dampening, so that any friction caused by contact with the distal end of the velocity transformer 30 does not result in unnecessary friction-induced high pitched noise. The wick 76 is placed appropriately so that there is sufficient contact with the distal end of the velocity transformer 30, and allows for a capillary amount of vaping liquid to flow onto the distal end of the velocity transformer 30 for nebulization. As more of the liquid is nebulized, the wick 76 continues to draw more liquid 74 from the tank 72 to the velocity transformer 30. When the tank assembly 70 is removed from the nebulizer housing 12, the wick 76 acts as a stopper to prevent leakage of the vaping liquid 74. On another opening end of the tank 72, a stopper 78 plugs the opening and can be removed to allow refilling of the vaping liquid 74. The tank assembly 70 can thus be easily removed for cleaning or replaced inexpensively without affecting any components of the transducer assembly 20.

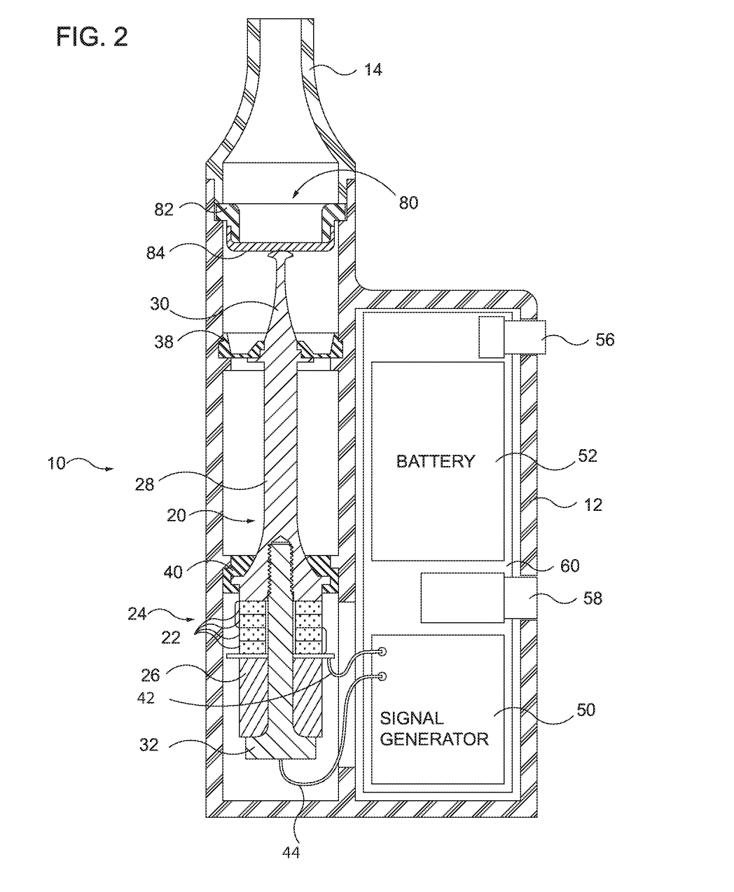

In FIG. 2 of the second embodiment, a removable container 80 is adapted between the mouthpiece 14 and nebulizer housing 12, and in contact with the distal end of the velocity transformer 30. The container 80 is constructed of a soft bottom membrane 84, and rigid side-wall 82. The soft bottom membrane 84 is made from resilient and pliable materials such as silicone or thermoplastic elastomer or any material that can withstand the vibrations of the velocity transformer 30 without melting or breaking, and at the same time acoustically dampening to prevent generating friction-induced noise when the transducer 20 is activated. Further, the thickness of the soft bottom membrane 84 should preferably be thin, about 0.5 mm (0.02″) so that vibratory energy can be transmitted from the velocity transformer 30 through the material of the bottom piece 84. The side-wall 82 is made from materials such as plastic, metal, glass or ceramic. The soft bottom 84 can be over-molded onto the side-wall 82, or it can be stretched over the side-wall 82 as a two-piece constructed tank 80.

Whichever method the tank 80 is constructed, the soft bottom membrane 84 should preferably be taut around the side-wall 82, so that when the bottom 80 is in contact with the velocity transformer 30, vibratory energy can be efficiently transferred to the content of container 80. Vaping liquid or solid placed inside the container 80 can thus be energized by the velocity transformer 30 for nebulization without being in direct contact with the velocity transformer 30. Such vaping liquid or solid used in this embodiment are typically gel-like or wax-like with high viscosity and do not flow freely through a wick 76 as described in FIG. 1. The container 80 described in this embodiment can thus be easily removed for cleaning or replaced inexpensively without affecting any components of the transducer assembly 20.

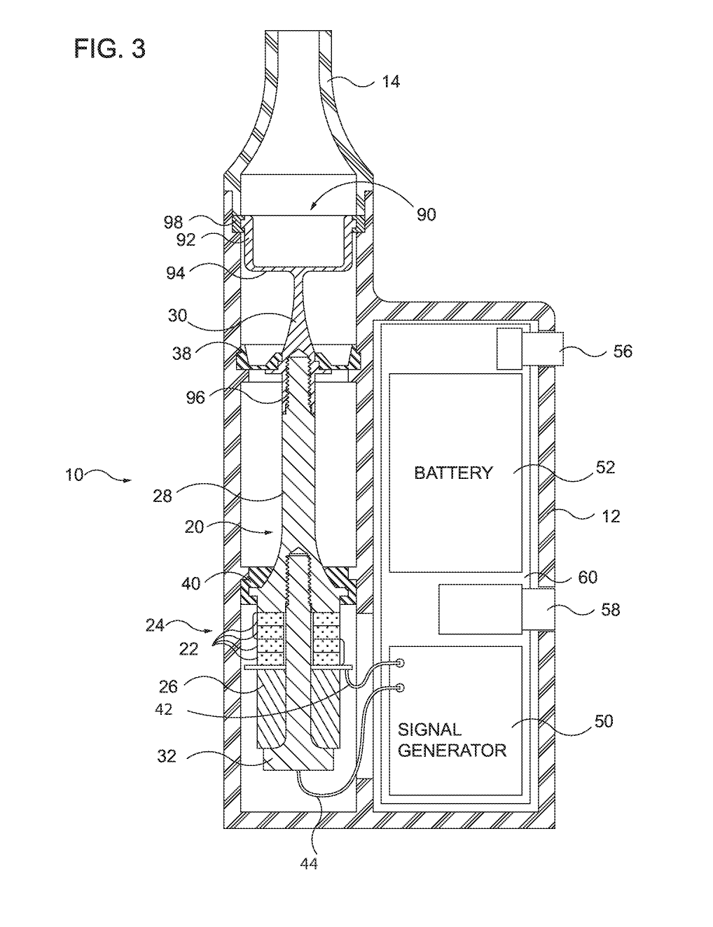

In FIG. 3 of the third embodiment, a solid container 90 is integrated as part of the velocity transformer 30. The velocity transformer 30 has a first end and a second end. The sonotrode 28 comprises a first end coupled to the transduction section 24 of the transducer 20. The second end of the sonotrode 28 has a threaded distal end coupled to the first end of the velocity transformer 30. The second end of the velocity transformer has an attached container 90, comprising a bottom 94 and side-wall 92. An acoustic isolator 98 is adapted between the container 90 and nebulizer housing 12 to minimize any transfer of vibration and friction to the housing 12. The velocity transformer 30 and attached container 90 can thus be removed from the sonotrode 28 by unscrewing from the sonotrode 28 for cleaning or storage. When the velocity transformer 30 is coupled to the sonotrode 28, ultrasonic energy from the acoustic assembly 20 is transferred to the bottom 94 of the container 90. Vaping liquids or solids placed inside the container 90 can thus be energized by the velocity transformer 30 for nebulization. Such vaping liquid or solid used in this embodiment are typically gel-like or wax-like with high viscosity and do not flow freely through a wick 76 as described in FIG. 1.

The container 90 and the attached velocity transformer 30 can be made from a single metallic material such as titanium, stainless steel or aluminum. Alternatively, the container 90 and velocity transformer 30 are separate components attached together with glue or fasteners. In either case, the container 90 and velocity transformer 30 should be attached in such a manner as to allow the maximum transfer of ultrasonic energy from the acoustic assembly 20 to the bottom 94 of the tank 90, without looseness or allowing for friction between the container 90 and velocity transformer 94. Any looseness or friction will result in audible and undesired high-pitched friction noise. Transducers are currently available and are used in several devices like dental scalers and surgical knives, and existing devices are shown and described in the following patents: U.S. Pat. No. 6,278,218; U.S. Pat. No. 5,702,360; and U.S. Pat. No. 8,257,377.

There are 3 improvements or embodiments of the design. The first one is for liquid and are the most applicable for e-cigarettes. The other two are more suitable for concentrates and THC waxes.

### Embodiment 1

### For Liquids

For the first embodiment, the most important component or method is the delivery of the liquid from the tank to the transducer using a wick. No ultrasonic nebulizers for medicine use a wick; most of them have transducers, which are attached directly to a container to fill the liquid. Using a wick limits the amount of liquid to the transducer, such that it is just enough for atomization. Using a container like existing nebulizers requires the transducer has to be so powerful as to transfer energy through the depth of the liquid, but with instant improved design, less power is required.

### Embodiment 2

For the second embodiment, using silicone to atomize THC is a big discovery because the energy from the transducer can pass through silicone to melt and then atomize the THC, but it is important to have the silicone material taut.

Note that hard materials will not work, such as metal, glass or ceramic; the transducer scratches the material and creates an audible screeching sound. If in plastic, the transducer actually melts and cut through the plastic, much like an ultrasonic welding machine. Silicone has another advantage because THC material does not stick to the silicone and makes for easier cleaning.

### Embodiment 3

The third embodiment improves on Embodiment 2 (silicone) and has a metal container that is attached to the transducer so there is as little as possible friction between the two parts; little or no friction results in reduced or no sound.

The function of a typical nebulizer is directly related to the amount of liquid above the probe (or velocity transformer) of the transducer. The thicker the amount of liquid above the probe, the more powerful the transducer needs to be in order to transmit the acoustic energy to the surface of the liquid to cause atomization.

To minimize the amount of power required, a thin film of liquid is desired, so that less energy (thus smaller battery) is sufficient to atomize the liquid; the preferred wick will draw just enough liquid to the probe so that no big puddle is formed on the probe (i.e., big puddle=thick amount=insufficient energy to transmit through the thickness of the liquid).

Conversely, if the wick is too dense, not enough liquid is drawn to the probe for optimal vapor output. The liquid flow amount of the wick can be controlled by the wick's density, porosity, and/or constricting the wick's diameter.

Detailed embodiments of the present invention are disclosed; however, the disclosed embodiments are merely exemplary of the invention, which can be embodied in various forms; specific structural and functional details disclosed are not to be interpreted as limiting, but merely as a basis for the claims and as a representative basis for teaching one skilled in the art to variously employ the present invention in virtually any appropriately detailed structure. The title, headings, terms and phrases used are not intended to limit the subject matter or scope; but rather, to provide an understandable description of the invention. The invention is composed of several sub-parts that serve a portion of the total functionality of the invention independently and contribute to system level functionality when combined with other parts of the invention. The terms “a” or “an” are defined as: one or more than one. The term “plurality” is defined as: two or more than two. The term “another” is defined as: at least a second or more. The terms “including” and/or “having” are defined as comprising (i.e., open language). The term “coupled” is defined as connected, although not necessarily directly, and not necessarily mechanically.

Any element in a claim that does not explicitly state “means for” performing a specific function, or “step for” performing a specific function, is not be interpreted as a “means” or “step” clause as specified in 35 U.S.C. Sec. 112, Paragraph 6. In particular, the use of “step of” in the claims herein is not intended to invoke the provisions of 35 U.S.C. Sec. 112, Paragraph 6.

Incorporation by Reference: All publications, patents, patent applications and Internet website addresses mentioned in this specification are incorporated by reference to the same extent as if each individual publication, patent, or patent application was specifically and individually indicated to be incorporated by reference; U.S. Patent Appl. 61/921,906, filed on Dec. 30, 2013; 61/928,823, filed on Jan. 17, 2014; 61/928,797, filed on Jan. 17, 2014; Ser. No. 14/271,442, filed on May 6, 2014; and Ser. No. 14/272,414, filed on May 7, 2014; U.S. Pat. No. 6,278,218; U.S. Pat. No. 5,702,360; U.S. Pat. No. 8,257,377; U.S. Pat. No. 6,325,811; U.S. Pat. No. 5,954,736, which are all incorporated by reference in entirety.

I claim:

1. An apparatus for ultrasonic atomizing of a liquid in a container, comprising:
a housing with a power source;
the housing has a mouthpiece and the container with the liquid for atomizing;
the container has a wick for drawing the liquid to a probe on an ultrasonic transducer, which is located in the housing;
the probe engages the wick and allows for a displacement of greater than 10 micrometer;
the ultrasonic transducer is connected to the power source and a signal generator;
Whereby when activated, the ultrasonic transducer vibrates the probe, such that the liquid from the wick is atomized, and vapor of the atomized liquid exits the mouthpiece.

2. The apparatus of claim 1 wherein the power source is an internal battery or an external power source; and the ultrasonic transducer is activated by an activation switch or an activation button.

3. The apparatus of claim 1 wherein the container is removable from the housing.

4. The apparatus of claim 1 wherein the ultrasonic transducer has multiple piezoelectric elements, a transduction portion, an anvil bearing member and a sonotrode amplification member; and the ultrasonic transducer is acoustically coupled.

5. The apparatus of claim 1 wherein the housing has acoustic isolators to dampen vibrations emitted from the ultrasonic transducer to the housing.

6. An apparatus for ultrasonic atomizing of a substance in a container with a soft bottom membrane and at least one rigid side wall, comprising:
a housing with a power source;
the housing has a mouthpiece and the container with the substance for atomizing;
the housing has an ultrasonic transducer, which contacts the soft bottom membrane of the container;
the ultrasonic transducer is connected to the power source and a signal generator;
Whereby when activated, the ultrasonic transducer vibrates the soft bottom membrane of the container such that the substance is atomized, and vapor of the atomized substance exits the mouthpiece.

7. The apparatus of claim 6 wherein the power source is an internal battery or an external power source; and the ultrasonic transducer is activated by an activation switch or an activation button.

8. The apparatus of claim 6 wherein the ultrasonic transducer has multiple piezoelectric elements, a transduction portion, an anvil bearing member and a sonotrode amplification member; and the ultrasonic transducer is acoustically coupled.

9. The apparatus of claim 6 wherein the housing has acoustic isolators to dampen vibrations emitted from the ultrasonic transducer to the housing.

10. An apparatus for ultrasonic atomizing of a substance in a container, comprising:
a housing with a power source;
the housing has a mouthpiece and the container with the substance for atomizing;
the housing has an ultrasonic transducer, which is integrated with the container;
the ultrasonic transducer is connected to the power source and a signal generator;
Whereby when activated, the ultrasonic transducer vibrates the container such that the substance is atomized, and vapor of the atomized substance exits the mouthpiece.

11. The apparatus of claim 10 wherein the power source is an internal battery or an external power source; and the ultrasonic transducer is activated by an activation switch or an activation button.

12. The apparatus of claim 10 wherein the ultrasonic transducer has multiple piezoelectric elements, a transduction portion, an anvil bearing member and a sonotrode amplification member; the ultrasonic transducer is acoustically coupled.

13. The apparatus of claim 10 wherein the housing has acoustic isolators to dampen vibrations emitted from the ultrasonic transducer to the housing.
